(19) United States
(12) Patent Application Publication
(10) Pub. No.: US 2024/0423781 A1
(43) Pub. Date: Dec. 26, 2024

(54) Antony Frame

(71) Applicant: Anuja K Antony, Cary, NC (US)

(72) Inventor: Anuja K Antony, Cary, NC (US)

(21) Appl. No.: 18/237,914

(22) Filed: Aug. 25, 2023

Related U.S. Application Data
(60) Provisional application No. 63/403,289, filed on Sep. 1, 2022.

(51) Int. Cl. A61F 2/12; A61F 2/00

(52) U.S. Cl. CPC A61F 2/12; A61F 2/0063

(57) ABSTRACT

Described herein is a medical device mold for use in positioning a medical device in the body. The mold holds implantable medical devices such as breast implants in three dimensions thereby overcoming limitations associated with breast reconstruction and augmentation. Methods of use are also described herein.

### CROSS-REFERENCE TO RELATED APPLICATIONS

This application claims benefit under 35 U.S.C. § 119(e) of U.S. Provisional Application No. 63/403,289 filed Sep. 1, 2022. The above listed application is incorporated by reference herein in its entirety for all purposes.

### FIELD OF THE DISCLOSURE

The disclosure relates to a medical device positioner. The disclosure also relates to methods of using the medical device positioner in breast reconstruction and augmentation procedures.

### BACKGROUND

Breast reconstruction is a common restorative surgery performed after mastectomy. In addition, breast augmentation is a popular elective surgery, chosen to enhance and lift the look of the breasts and instill self-confidence. Common surgical options for breast reconstruction include the medical devices-breast implants and/or breast expander. In one option, the plastic surgeon uses a tissue expander to fill the defect created during the mastectomy. Saline is injected into the tissue expander at regular intervals in order to stretch the skin in preparation of a future exchange for a breast implant. The breast implant then remains in the body. The use of tissue expanders is a common practice, especially when a patient desires a larger breast. Removal of the breast (i.e mastectomy) and insertion of a tissue expander can be done in a single surgery. In a follow-up surgery the tissue expander is replaced with the implant.

Breast augmentation involves the use of saline or silicone implants which are positioned in the chest to enhance the appearance of the breast. Tissue expanders and breast implants are positioned in a chest pocket created via an incision in the overlying chest skin. The expander or implant is often wrapped in a dermal mesh or similar material for use in securing the implant in the chest and the inserted in the breast pocket while the patient is in the supine position. The patient is then positioned in the upright position and the aesthetics and positioning of the device evaluated. Upon return to the supine position the expander or implant is secured in a final anatomical position. Despite this, tissue expanders and implants commonly migrate from their intended position over time. This implant malposition is attributable to many factors including inadequate surgical technique, poor implant pocket construction, implant weight; lack of soft tissue support, chemotherapy, surgeon misunderstanding regarding the interaction between tissue expanders/breast implants and tissue healing dynamics, or inadequately securing the medical device in place. As a result, a follow-up surgery is often required to correct the implant. However, these surgeries incur excess costs for the patient and are often limited in effectiveness and options due to scar tissue and tissue healing dynamics that make follow-up surgery difficult.

Moreover, single stage prepectoral breast construction is a procedure in which during the same surgery, following mastectomy a final implant is placed, thereby eliminating the need for the tissue expander and additional surgeries. However, this is a highly complex and technically challenging surgery, so much so that reconstruction breast surgeons often elect to not offer their patients a single stage prepectoral surgery. The surgery is commonly associated with implant migration over time and scarring in the form of capsular contraction resulting in an aesthetically displeasing result and the need for one or more follow-up revisions or removal of the implant. Thus, despite the benefits of breast reconstruction there exists a need for a medical device that aids in positioning and securing the breast expander and/or implant in the chest pocket. The present application discloses a medical device positioner that would overcome challenges associated with placement and adherence of medical devices within the body, especially those challenges as they relate to breast reconstruction and augmentation.

### SUMMARY

The disclosure provides a medical device positioner that is used for positioning a medical device within the body. In a preferred embodiment the medical device positioner is used for positioning a breast implant or tissue expander. In the breast pocket following mastectomy.

In one aspect described herein is a method for positioning an implantable medical device in a body, wherein the implantable device is covered in a biologic mesh creating a wrapped device; surrounding the wrapped device with a first enclosure; mating the first enclosure with the wrapped device with a second enclosure that are then used to position the wrapped device within the body, accounting for the x, y, and z planes; adhering the implantable device covered in biologic mesh to the body; and removing the first and second enclosures from the body. In one embodiment of the method the first enclosure is adhered within the body using sutures, adhesive or clamps. Alternatively, the first enclosure is adhered in the body using attachment points integrated into the first enclosure. The first and second enclosures may be comprised of a biodegradable or absorbable material.

In one embodiment of the first and second enclosures are round and have different circumferences. In another aspect the first and second enclosures are spherical with a fixed circumference. The circumference of the first and second enclosures can be adjustable or fixed and the shapes of the enclosures can be the same or different. Exemplarily shapes of the first and second enclosures include circular, oval, square, rectangular, triangular, elliptical, or a modification thereof.

In yet another embodiment the first embodiment, the first and/or second enclosure has an attachment point that allows attachment of the enclosures to each other. The first or second enclosure may also have a single tab adhered to any edge of the enclosure. In an alternative embodiment the first or second enclosure contains a plurality of attachment points wherein the attachment points can be removed from the first and/or second enclosures, may be biodegradable or bioabsorbable.

In another embodiment the implantable device is a breast implant, a breast sizer, acellular dermal matrix, an absorbable, or a non-absorbable mesh and can include placing an overlying mold over the implantable device.

In another embodiment the implantable device (i.e. breast implant) is covered in biological mesh that is attached to either the first and/or second enclosure of a medical device positioner and the biological mesh covered breast implant secured in the breast pocket followed by removal of the medical device positioner from the human body. In a preferred embodiment the wrapping of the implantable device occurs outside of the body.

In another aspect is a structure configured to position and/or hold an implantable device in place in the body of a subject comprising a first enclosure surrounding an implantable device; a second enclosure that mates with the first enclosure; and wherein the structure is removable from the body. The structure can also comprise an overlying mold and is configured to surround an implantable device and hold the implantable device in the chest using an attachment point that secures the first enclosure to the second enclosure. The enclosures can be comprised of a non-biological or biological material.

In other embodiments the enclosures can be round and may have the same circumference although this is not necessary. The shape of the enclosures can be circular, oval, square, rectangular, triangular, elliptical, or a modification thereof.

In further embodiments the medical device positioner comprises a latch, hinge, or fastener. In some embodiments the attachment point comprises a tightening-type device, peg and hole system, interlocking pegs, hinge, or nested enclosures. The first and/or second enclosures may also contain attachment points that can are used to secure the enclosure in the body. The attachment points can be removed from the enclosure and may be biodegradable or bioabsorbable.

In another embodiment the implantable device is one or more of a breast implant, a breast sizer, acellular dermal matrix, or an absorbable or non-absorbable mesh.

Specific embodiments of the disclosure will become evident from the following more detailed description and the claims.

### BRIEF DESCRIPTION OF THE DRAWINGS

FIG. 1 is a schematic a medical device positioner and method of use in a patient. FIG. 1A is a superior view of a medical device positioner (1). The positioner has a first enclosure (1) and a second enclosure (2) in addition to a fastener mechanism (12). FIG. 1B is a medical device positioner enclosing a breast implant or similar medical device (2). FIG. 1C is an enclosure system (6) comprised of a medical device positioner (1) having a first enclosure (5) and second enclosure (4) and fastener system (12) enclosing a breast implant (2) wrapped in dermal mesh (3). FIG. 1D is a schematic of the enclosure system (6) as placed in the breast pocket of a patient. FIG. 1E is mesh wrapped breast implant (7) after adherence in the breast pocket and removal of the medical device positioner.

FIG. 2 is a schematic of medical device positioner. The positioner is comprised of a first enclosure ring or rod (8) that mates in a channel, track, or groove (9) of a second enclosure (4) thereby pinching the mesh (22) between enclosures ((8) and (9) FIG. 2A. The positioner can further comprise an optional fastener system (12) for use in tightening the positioner around the mesh covered breast implant. FIGS. 2B-2D are schematics of first enclosure mating with the second enclosure.

FIG. 3 is a schematic of medical device positioner. The positioner is comprised of a first enclosure (5) and second enclosure (4). FIG. 3A is a schematic of the mating system in which one or more pegs (10) in a first or second enclosures mate with a hole system (11) of a first of second enclosure. FIG. 3B is schematic of the mated medical device positioner.

FIG. 4 is a schematic of a medical device positioner. FIG. 4A is schematic of the positioner, which is comprised of first and second enclosures wherein a peg (10) and hole (11) mate and are further can be tightened around the mesh wrapped breast implant using tandem fastener (12). FIG. 4B is a schematic of the mating of the first and second enclosure. The first enclosure can have the peg or the hole and the second enclosure the other of the hole or the peg

FIG. 5 is a schematic of a medical device positioner. The positioner is comprised of a first (5) and second (4) enclosure that are interrupted. In this embodiment the first enclosure (4) fits into a groove, channel, or cutout (13) in the second (4) enclosure. The embodiment further has a pin (10) and hole (11) in the second enclosure (4) that allows for joining of the opposite ends. The peg can be fixed or varying lengths. In one embodiment the peg (10) is removable from the second enclosure allowing for use of a peg or a preferred length. An alternate embodiment utilizes multiple enclosures (20) that nest inside each other (FIG. 10). The enclosures can be non-continuous (20) with a fastener system (12), as described herein that is used to decrease or increase the enclosure space. In both embodiments the biological mesh (22) wrapping the breast implant is pinched between the enclosures or enclosure and the groove or channel thereby forming the mesh implant enclosure system. The enclosure system is placed in the breast pocket, and upon attachment of the mesh wrapped breast implant in the correct anatomical location the medical device positioner can be removed from the body.

FIG. 6 is a schematic of a medical device positioner wherein each of the first (5) and second (4) enclosures are continuous (i.e., not interrupted) and nest inside of each other. FIG. 6A is schematic of a first enclosure (5) having a top surface (14) that is the same shape but slightly wider than the remainder of the first enclosure. FIG. 6B is a schematic of the second enclosure and the mating of the first and second enclosures. The second enclosure (4) has a lip (13) that extends into the open space of the second enclosure. This allows the first enclosure to nest and interact with the second enclosure at the lip (13).

FIG. 7 is a schematic of medical device positioner. The positioner has a first enclosure (5) is smaller than the second enclosure (4) allowing for the first enclosure to reside within the diameter of the second enclosure. Each of the first and second enclosures are non-continuous. The first and second enclosures are secured to each other via a peg (10) and hole (11) system wherein the peg (10) is on the inside lateral surface of the second enclosure (4) or the outside lateral surface of the first enclosure (5).

FIG. 8 is a schematic of a medical device positioner. FIG. 8A demonstrates a positioner comprised of a single enclosure (15) wherein a fastener is embedded in the entire length of the interior of the enclosure (16). FIG. 8B demonstrates that in this embodiment the single enclosure surrounds the medical device, such as a breast implant covered in biological mesh (3). The enclosure further contains biodegradable tabs (17) for securing the mesh wrapped breast implant in the correct anatomical location.

FIG. 9 is a schematic of a medical device positioner. FIG. 9B demonstrations that the positioner has spikes (19) in the first enclosure (5) that interact with the holes of the second enclosure (4) (FIG. 9A). The spike into hole secures the mesh wrapping the breast implant.

FIG. 10 is a schematic of medical device positioner. The positioner utilizes multiple enclosures (20) that nest inside each other. The enclosures can be non-continuous (20) with a fastener system (12), as described herein. The fastener is used to decrease or increase the enclosure space. The biological mesh (22) wrapping the breast implant is pinched between the enclosures or enclosure and the groove or channel thereby forming the mesh implant enclosure system.

FIG. 11 is a schematic of medical device positioner wherein a first and second enclosure are mated. The mesh (22) is pinched between the first and second enclosures. The design allows for suturing inside enclosures.

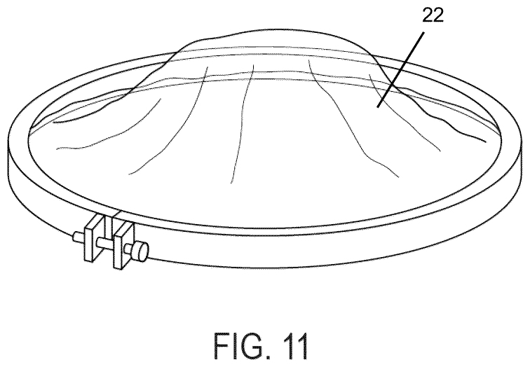

FIG. 12 is a schematic of medical device positioner (1). FIG. 12A demonstrates the medical device positioner having a first (5) and second (4) enclosure. FIG. 12B is a schematic of a breast implant (2) within the positioner FIG. 12C is a schematic of the biological mesh (3) placed over the breast implant.

FIG. 13 is a (medical device positioner. FIG. 13A demonstrates the medical device positioner comprised of a first enclosure (5) and second enclosure (4) wherein the second enclosure is located within the first enclosure. The second enclosure contains rod or pegs (25) that extend through the first enclosure. The mesh associated with the mesh coated breast implant falls into the space between the first and second enclosures (23) and a plurality of screws, sliders, or other tightening mechanisms (24) are tightened against the first enclosure, thereby pinching the biological mesh (2) FIG. 13B demonstrates a mesh (2) wrapped breast implant in the medical device positioner. FIG. 13C demonstrates that the second enclosure further comprises a basement membrane tab system (27) between the first and second enclosures. FIG. 13D demonstrates the mesh enclosure system with the mesh covered implant and medical device positioner.

### DETAILED DESCRIPTION

The disclosure relates to a medical device positioner for positioning a medical device within the body. More particularly, the disclosure relates to a medical device for use in positioning and holding breast implants and/or tissue expanders in place in the breast pocket thereby preventing migration of the breast implant following surgery. The disclosure provides surgeons a device and method by which to overcome the challenges that have plagued single stage pre-pectoral breast reconstruction.

Breast augmentation can be undertaken for aesthetic reasons, reconstructive reasons, or both. Individuals with breast cancer undergo either a single or double mastectomy followed by plastic surgery to reconstruct the removed tissue. A variety of surgical methods exist for breast reconstruction. A common reconstruction method is placement of a tissue expander in place of the excised tissue. A tissue expander is a saline-filled medical device placed in the breast pocket; the space that remains once breast tissue has been excised. The tissue expander volume is increased by adding additional saline to the device on a regular basis. As the device expands it stretches the overlying skin, the goal is to stretch the breast skin to accommodate the desired size of the final implant. The tissue expander is placed, following, but during the same surgical session, as the mastectomy. In a follow-up surgery the expander is exchanged for the final breast implant.

The breast implant can be saline or silicon-filled and comes in a variety of textures, shapes and sizes. The implant exchange often serves as the final surgery in the breast reconstruction journey. However, a more desirable surgery is a single stage prepectoral breast reconstruction wherein in the same surgery, following the mastectomy, the final implant is placed in the breast pocket, above the pectoralis muscle. This placement is aesthetically more pleasing than placing the implant under the pectoralis muscle. However, the technique is technically difficult because the implant migrates from the intended position over time.

The medical device positioner of the current disclosure provides the advantageous technical effect of positioning a breast implant, tissue expander, or other medical device within the human body, accounting for x, y, and z planes (i.e., three-dimensional) thereby preventing unintended movement and migration of the device prior to final adherence in the intended space, when compared to currently used methods for breast reconstruction and augmentation. This reduces the number of surgeries, is associated with reduced pain, and improved patient satisfaction.

Reference will now be made in detail to exemplary embodiments of the claimed invention. While the claimed invention will be described in conjunction with the exemplary embodiments, it will be understood that it is not intended to limit the claimed invention to those embodiments. To the contrary, it is intended to cover alternatives, modifications, and equivalents, as may be included within the spirit and scope of the claimed invention, as defined by the appended claims.

Those of ordinary skill in the art may make modifications and variations to the embodiments described herein without departing from the spirit or scope of the claimed invention. In addition, although certain methods and materials are described herein, other methods and materials that are similar or equivalent to those described herein can also be used to practice the claimed invention.

### 1. Definitions

Unless defined otherwise, all technical and scientific terms used herein have the meaning commonly understood by a person skilled in the art to which the claimed invention belongs. The terminology used herein is for describing particular embodiments only and is not intended to be limiting of the claimed invention. All technical and scientific terms used herein have the same meaning. Preferred methods and materials of the preferred disclosure are described herein. However equivalent and/or alternative methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

As used herein, the singular forms “a,” “and,” and “the” include plural references, unless the context clearly dictates otherwise.

As used herein, the term “or” means, and is used interchangeably with, the term “and/or,” unless context clearly indicates otherwise.

As used herein, the term “including” means, and is used interchangeably with, the phrase “including but not limited to.”

As used herein, the term “such as” means, and is used interchangeably with, the phrase “such as, for example” or “such as but not limited.”

As used herein, the terms “comprise(s),” “include(s),” “having,” “has,” “can,” and “contain(s),” as used herein, are intended to be open-ended transitional terms. The terms do not preclude the possibility of additional acts or structures.

As used herein the disclosure also contemplates other embodiments through use of “comprising,” “consisting of’ and “consisting essentially of,” the embodiments or elements presented herein, whether explicitly set forth or not.

Unless specifically stated or obvious from context, as used herein, the term “about” is understood as within a range of normal tolerance in the art, for example, within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, “subject” and “patient” are used interchangeably to refer to any vertebrate, including, but not limited to, a mammal, non-human primate including but not limited to a cynomolgus monkey, rhesus monkey, chimpanzee, or similar). In the preferred embodiment the terms refer to a human subject or human patient. The subject or patient can be healthy or non-healthy or undergoing or have undergone various forms of treatment.

As used herein the term “biocompatible” has the same meaning as “biocompatibility”. The terms refer to a material or a property of a material that does not cause an adverse reaction or has properties only associated with minimal adverse reactions in a subject, preferably a human subject, when placed in proximity to a subject or subject's body tissues or when in internal or external contact with one or more tissues from a subject. The term “biodegradable mesh” refers to a medical device scaffold that supports damaged tissue. Biodegradable meshes are intended to support, elevate, and reinforce soft tissue repairs. The mesh can be synthetic or biologic and can also be acellular. Biodegradable meshes can be animal derived such as from swine or other animals. In a preferred embodiment the mesh is human derived. The biodegradable mesh can also be synthetic, developed in a laboratory.

As used herein, the terms “biodegradable” and “bioabsorbable” refer to a material or a property of a material capable of being catabolized, metabolized, and/or absorbed by a subject inside a subject by any means, without resulting in significant adverse reaction in a subject. Biodegradable material and a device made of biodegradable material are substantially absorbed or broken down by in the body without having significant adverse physiological and/or biochemical effects on the subject.

As used herein, “implant” and “implantable device” generally refer to an article or device that is placed entirely or partially into a subject (e.g., breast implant), for example by a surgical procedure or medical intervention, for any period of time.

As used herein, the term “medical device positioner” refers to a medical device that is used to position, hold, orient, configure, temporarily secure, or permanently secure a second medical device within the human body. The medical device positioner can be permanent or removable. Removable devices can be made of materials, including but not limited to metals, alloys, ceramic, silicone, rubber, plastic, or ceramic. Permanent positioners can be made of one or materials such as, but not limited to polyethylene, polyurethane, polylactic acid, polydioxanone, polyglycolide, polyglycolic acid, polyglactin, polyglyconate, lactomer glycolide/lactide copolymer, polydimethylsiloxane, polysiloxane, polypropylene, caprolactone, poliglecaprone, polycaprolate, or combinations thereof. In preferred embodiments the medical device positioner aids in positioning and securing a breast prosthetic, such as an implantable device in the chest cavity. In one embodiment the medical device positioner can be used in a prepectoral breast construction surgery. In a preferred embodiment the medical device positioner is comprised of one or more enclosures and an optional fastener. The positioner is novel as current methods of securing breast implants only position the breast in the x and y planes, thereby failing to represent the aesthetics, gravitational, and physiological components of the z plane. The medical device positioners described herein are technical superior to existing products that only position the breast implant in the x and y planes. Therefore, the results obtained in during surgery are often not representative of the final implant position. The positioners described herein solve the technical need for a device that positions the breast in all three planes while also overcoming the technical hurdles that have plagued pre-pectoral breast implant surgeries such as poor aesthetic outcomes and inadequate understanding of the prepectoral breast implants in the field that has discouraged use of direct to implant techniques (i.e., skipping the tissue expander surgery). As a result, reconstructive surgeons often resort to placing implants under the pectoralis muscle and/or performing a multi-step surgery wherein a breast expander is inserted at the time of mastectomy, and the expander exchanged for a breast implant in a separate surgery weeks later. Thus, the patient incurs multiple surgeries, excess downtime, and excess costs when compared to a direct to implant prepectoral surgery using the medical device positioner as described herein.

As used herein, the term “breast pocket” refers to an opening in the chest into which the implantable medical device is secured. The breast pocket is anatomically located to allow for placement of the medical device positioner and eventually the implant in position that is aesthetically appropriate. The breast pocket location can be subglandular, subfascial, partially retropectrolly, totally submuscular or dual plane.

As used herein “enclosure system” refers to the medical device positioners that are used to hold an implantable medical device, such as a mesh wrapped breast implant in place. The term refers to a system comprised of a medical device positioner, the mesh wrapped breast implant or other medical device, and any accessory items such as tabs or latches. The enclosure system can be comprised of one or multiple enclosures in a variety of shapes as described herein as well as the mesh coated breast implant or other medical device. The enclosure system may also have a latch system or other mechanism for tightening the enclosures. In some embodiments the enclosures interface with each other thereby allowing for placement and tethering of the medical device in the desired location. In a preferred embodiment the enclosure system is placed in the breast pocket, positioned in the desired location and the biological mesh (covering the breast implant) sutured into place. The medical device positioner is then removed from the breast pocket and disposed or sterilized for re-use. The medical device positioner can also be manufactured from biodegradable material, in which case it is left in the breast pocket and dissolves over time.

As used herein “mesh wrapped breast implant” refers to an implantable medical device such as a silicone or saline breast implant that is wrapped with a biological mesh. In a preferred embodiment the implant is wrapped using an acellular dermal matrix. The implant, wrapped in mesh is enclosed in one or more enclosures of the medical device positioner, as described herein, thereby forming an enclosure system. The enclosure system is placed in the breast pocket, the mesh wrapped breast implant secured in correct anatomical location, and the medical device positioner removed from the body. In alternative embodiments the medical device positioner is made of biodegradable material and can be left in the body to dissolve.

### 2. Medical Device Positioner

The present disclosure relates to a medical device positioner. In the preferred embodiment the medical device positioner is configured to position, secure, and/or hold an implantable device in place in the body of a subject. In the preferred embodiment the positioner is used to position a breast implant and is comprised of a first enclosure surrounding an implantable device and a second enclosure that mates with the first enclosure. In a preferred embodiment the medical device positioner is intended for removal from the chest after the breast implant or tissue expander is secured in place. However, the positioner could be used at any location in the body where a medical device is intended to be placed.

The enclosures can be of a variety of sizes and shapes. In a preferred embodiment the enclosures are round. Round enclosures can be of any circumference and are dictated by the anatomical region and size of the space in which the enclosure is to be used. The medical device positioner can be comprised of a single enclosure or comprised of more than one enclosure. The enclosures can be round with a fixed circumference, alternatively the enclosures can be constructed wherein the circumference can be adjusted. However, in some embodiments the medical device positioner can be comprised of more than one device. The one or more enclosures can be the same circumference and in alternate embodiments the enclosures are circular, oval, square, rectangular, triangular, elliptical, or a modification thereof. In one embodiment the enclosures are designed in manner that allow for mating of the first and second enclosures. The first and second enclosures can be the same or different circumferences.

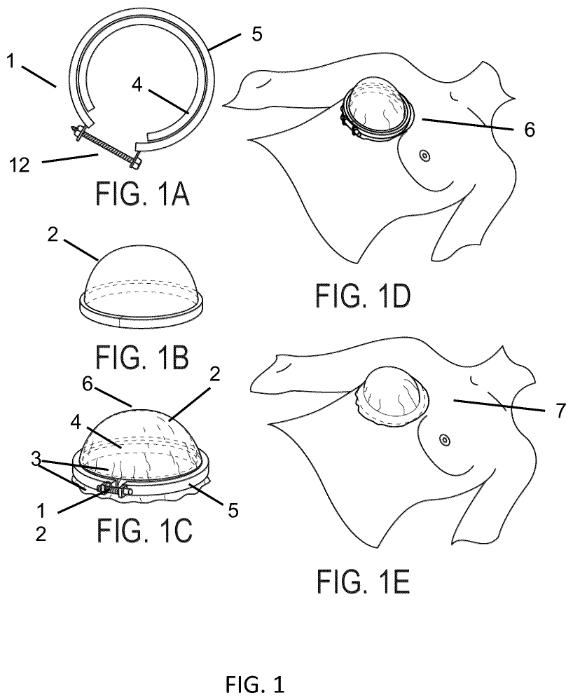

In a preferred embodiment of the medical device positioner, as shown in FIG. 1, a medical device positioner (1) is used to hold a breast implant or tissue expander in place. The positioner is comprised of a first enclosure (5) and second enclosure (4) with an optional fastener, hinge or latch mechanism (12). Each of the first (5) and second enclosures (4) can be continuous or interrupted, allowing for modifying the area encompassed by the medical device positioner through adjustment of the fastener or latch. The optional fastener can be a component of one or both of the first and second enclosures allowing for adjustments in the size of the enclosure. The fastener can be a bolt, pin, rod, screw, clip, latch, washer, anchor, u-bolt, key, or tie. The fastener can also be a tightening-type device.

In one embodiment, such as shown in FIG. 1, an implantable medical device such as a breast implant (2), is wrapped with a biodegradable or disposable mesh (3). The medical device positioner (1) surrounds the mesh wrapped breast implant or another implantable medical device (2). The second enclosure (4) of the medical device positioner mates with the first enclosure (5) to secure the mesh-wrapped breast implant in place (2) thereby creating an enclosure system (6) comprised of the mesh wrapped breast implant enclosed or held in place by the medical device positioners. The enclosure system is then positioned within the breast pocket (FIG. 1D). Final positioning of the implant and suturing of the mesh wrapped breast implant (3) are performed by the surgeon. The medical device positioner (1) is then removed from the breast pocket leaving the mesh wrapped breast implant or medical device (7) in the appropriate anatomical position (FIG. 1E).

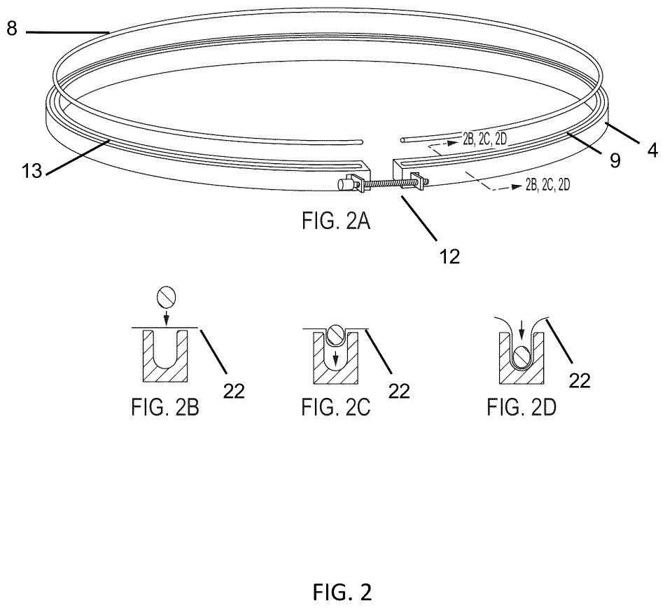

The medical device positioner is comprised of enclosures wherein the second enclosure (4) mates with the first enclosure (5) via variety of formats. In one embodiment the enclosures nest within each other ((1) FIG. 1A). In another embodiment a first enclosure rod or ring ((8) FIG. 2A) resides in a channel, track, or groove (5) of a second enclosure (9) thereby pinching the mesh (22) between the enclosures (4) and (5) to secure it between the enclosures (FIGS. 2A-2D). The rod or ring (8) is designed such that it fits into the groove of the second enclosure, regardless of shape, thickness, or overall design. The medical device positioner described herein can have enclosures that are continuous or non-continuous. A continuous enclosure is uninterrupted, or a single continuous enclosure of any particular shape with no distinct ends. Alternatively, a non-continuous or interrupted enclosure has two distinct ends.

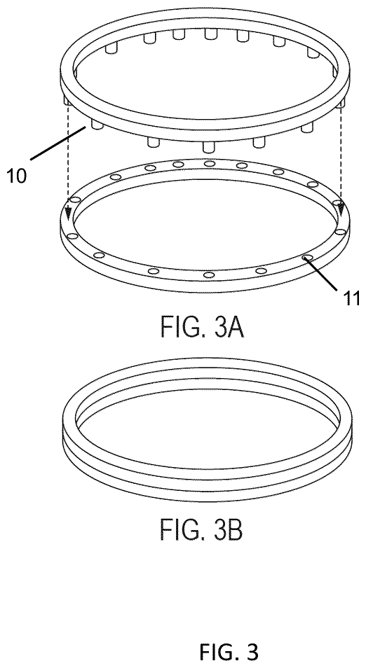

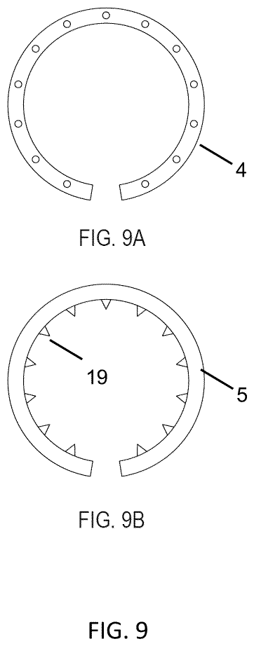

In yet another embodiment the enclosures mate with each other using a peg (10) and hole system ((11); FIG. 3A) or interlocking peg system wherein the enclosures have a top and bottom surface. The pegs (10) protrude from either the top surface or bottom surface of the first or second enclosure. The holes (11) can completely or partially penetrate the top or bottom surface of the first or second enclosure. The pegs and holes are of a size that allows for mating of the pegs and thereby the two enclosures. The number of pegs and holes can vary and the number of holes and pegs to not have to be equal. The top and bottom enclosures are manufactured in a variety of thicknesses, diameters, and shapes. In a preferred embodiment the enclosures are oval and comprised of two enclosures. In a further embodiment, spikes (19) can be used in place of pegs (FIG. 9). In such an embodiment the first enclosure (4) and second enclosure (5) can be continuous or interrupted. In a preferred embodiment the enclosures are interrupted and nest with each other via the spikes and holes to secure the mesh wrapped breast implant in place thereby forming the enclosure system. More particularly, the mesh is placed between the first and second enclosures thereby securing the mesh wrapped breast implant or tissue expander. The spikes can be of varying lengths and shapes. The enclosure system is placed in the breast pocket, and upon attachment of the mesh wrapped breast implant in the correct anatomical location the medical device positioner can be removed from the body.

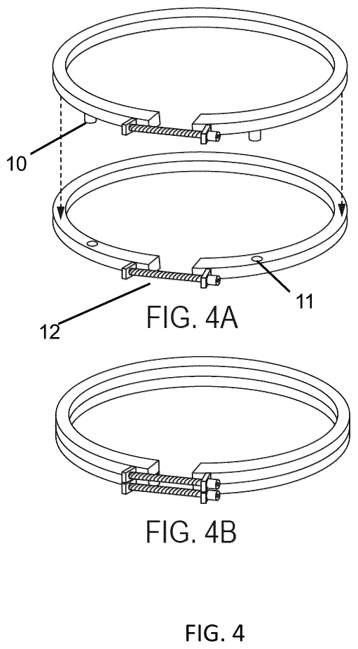

In another embodiment the first and second enclosures are comprised of a peg (10) and hole (11) system in tandem with a fastener (12) FIG. 4)). The fastener can be a bolt, pin, rod, screw, clip, latch, washer, anchor, u-bolt, key, or tie. The fastener is disposable for a single use or made from a material capable of being sterilized and reused. In some embodiments there may be a bolt only on the peg (10) or hole (11) enclosure. In other embodiments both the peg (10) and hole (11) have a bolt or pin (12).

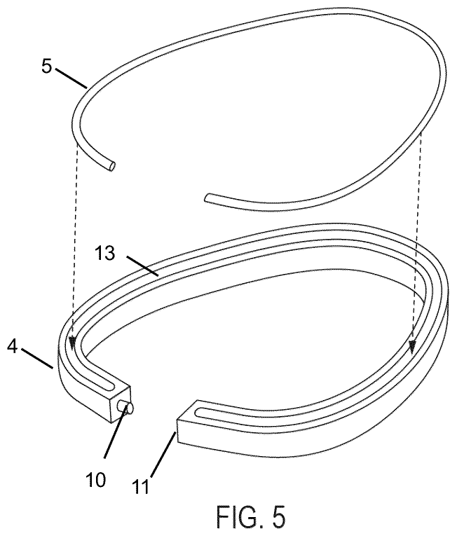

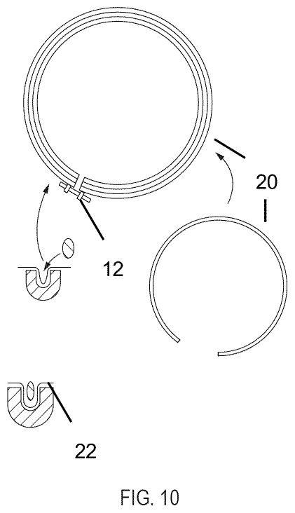

In yet another embodiment (FIG. 5) the first (5) and second (4) enclosures are interrupted. In this embodiment the first enclosure (5) fits into a groove, channel, or cutout (13) in the second (4) enclosure. The embodiment further has a pin (10) and hole (11) in the second enclosure (4) that allows for joining of the opposite ends of the enclosure. The peg can be fixed or varying lengths. In one embodiment the peg (10) is removable from the second enclosure allowing for use of a peg or a preferred length. An alternate embodiment utilizes multiple enclosures (20) that nest inside each other (FIG. 10). The enclosures can be non-continuous (20) with a fastener system (12), as described herein that is used to decrease or increase the enclosure space. In both embodiments the biological mesh (22) wrapping the breast implant is pinched between the enclosures or enclosure and the groove or channel thereby forming the mesh implant enclosure system. The enclosure system is placed in the breast pocket, and upon attachment of the mesh wrapped breast implant in the correct anatomical location the medical device positioner can be removed from the body.

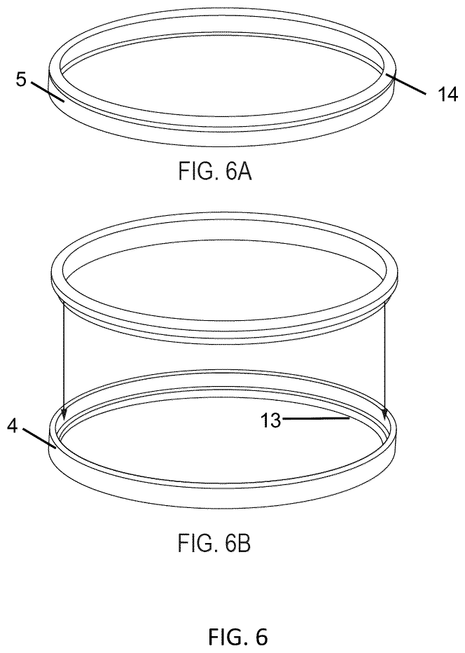

In another embodiment (FIG. 6) each of the first (5) and second (4) enclosures are continuous (i.e., not interrupted) and nest inside of each other. In this embodiment the second enclosure (4) has a lip (13) that extends into the open space of the second enclosure. The first enclosure (5; FIG. 6A) has a top surface that is the same shape but slightly wider (14) than the remainder of the first enclosure. This allows the first enclosure to nest and interact with the second enclosure at the lip (13). In this system the biological mesh of the mesh wrapped tissue expander is pinched between the first and second enclosures. The enclosure system is placed in the breast pocket, and upon attachment of the mesh wrapped breast implant in the correct anatomical location the medical device positioner can be removed from the body.

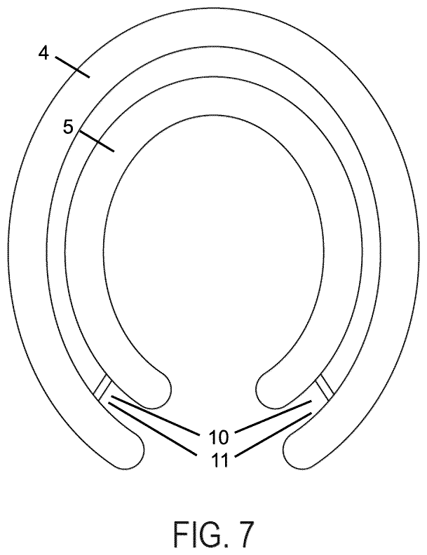

In another embodiment (FIG. 7) the first enclosure (5) is smaller than the second enclosure (4) allowing for the first enclosure to reside within the diameter of the second enclosure. Each of the first and second enclosures are non-continuous. The first and second enclosures are secured to each other via a peg (10) and hole (11) system wherein the peg (10) is on the inside lateral surface of the second enclosure (4) or the outside lateral surface of the first enclosure (5). The face opposite the peg contains a corresponding hole for mating with the peg. In this system the biological mesh covering the breast implant is inserted between the first and second enclosures with the peg penetrating the mesh. The enclosure system is placed in the breast pocket, and upon attachment of the mesh wrapped breast implant in the correct anatomical location the medical device positioner can be removed from the body.

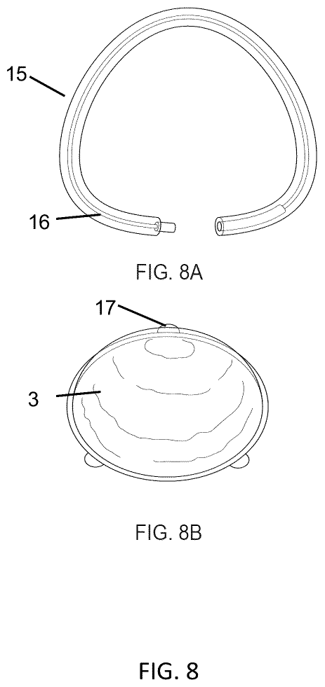

In another embodiment (FIG. 8A) is a medical device positioner comprising a single enclosure (15) wherein a fastener embedded in the entire length of the interior of the enclosure (16). In this embodiment the single enclosure surrounds the medical device, such as a breast implant covered in biological mesh (3). The enclosure further contains biodegradable tabs (17) for securing the mesh wrapped breast implant in the correct anatomical location. Upon attaching the mesh wrapped breast implant or other medical device in place using the tabs (17) the medical device positioner can be removed from the body.

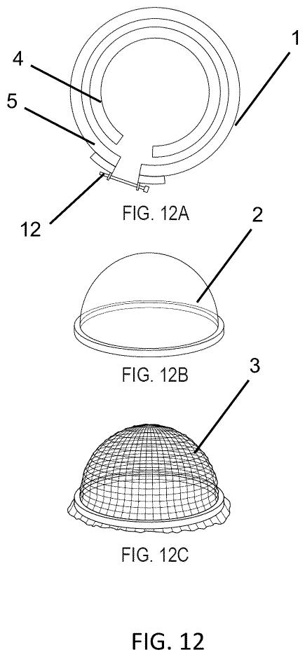

In another embodiment of the current disclosure (FIG. 12) a breast implant ((FIG. 12B; (2) is placed within a medical device positioner ((FIG. 12A (1). Any of the medical device positioners described herein can be used. The biological mesh is then placed over the breast implant (FIG. 12C). Upon attaching the mesh wrapped breast implant or other medical device in place the medical device positioner can be removed from the body.

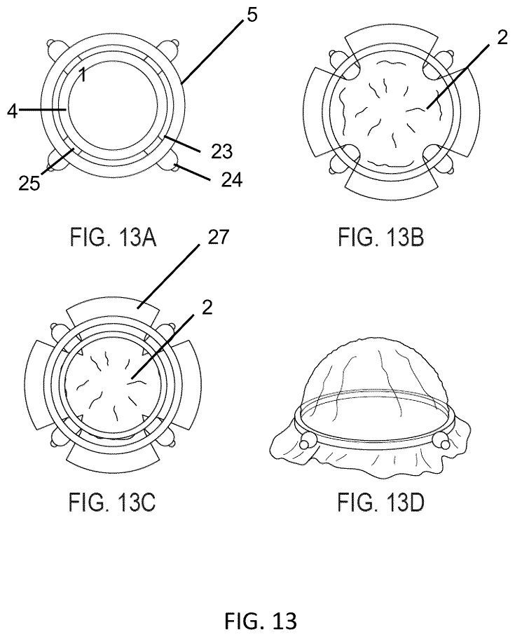

In another embodiment of the current disclosure (FIG. 13A) the medical device positioner is comprised of a first enclosure (5) and second enclosure (4) wherein the second enclosure is located within the first enclosure. The second enclosure contains rod or pegs (25) that extend through the first enclosure. The second enclosure contains a basement membrane tab system (27) The mesh associated with the mesh coated breast implant falls into the space between the first and second enclosures (23) and a plurality of screws, sliders, or other tightening mechanisms (24) are tightened against the first enclosure, thereby pinching the biological mesh (2) FIG. 13B demonstrates a mesh (2) wrapped breast implant in the medical device positioner. (FIG. 13B, superior view; FIG. 13C inferior view). In this embodiment upon attaching the mesh wrapped breast implant or other medical device in place the tabs (27) on the medical device positioner are cut and the positioner removed from the body. The tabs are further advantageous in providing flexibility and movement of the enclosure system. FIG. 13D demonstrates the mesh enclosure system with the mesh covered implant and medical device positioner.

### 3. Method of Positioning Implantable Devices

The present disclosure further relates to a method for positioning an implantable medical device in a body, wherein the implantable device is covered in a biologic mesh thereby wrapped device. The wrapped medical device is used in conjunction with a medical device positioner to form an enclosure system as described herein and positioning the system within the body. In a preferred embodiment the implantable device is a breast implant,. In other embodiments the device may be a tissue expander, or other medical device.

In the preferred embodiment the medical device positioner is configured to position, secure, and/or hold an implantable device in place in the body of a subject. In the preferred embodiment the positioner is comprised of a first enclosure surrounding an implantable device and a second enclosure that mates with the first enclosure. In a preferred embodiment the medical device positioner is intended for removal from the chest after the breast implant or tissue expander is secured in place. However, the positioner could be used at any location in the body where a medical device is intended to be placed.

The implantable device is wrapped using biological mesh, in a preferred embodiment the mesh is acellular dermal matrix, and the implantable device is a breast implant. The preferred implantable device is a breast implant. The enclosure system is comprised of the medical device positioner and mesh wrapped breast implant. Upon attaching the mesh wrapped breast implant or other medical device in the body, the medical device positioner can be removed from the body.

1. A medical device positioner configured to position and/or hold an implantable device in place in the body of a subject during a surgical procedure, comprising:
a first enclosure surrounding an implantable device;
a second enclosure that mates with the first enclosure;
wherein the first, second, or first and second enclosures can be adhered in the body thereby holding the implantable device in an anatomically desired position; and
wherein optionally the positioner is removed from the body following the surgical procedure.

2. The medical device positioner of claims 1 further comprising an overlying mold that is attached to the first or second enclosure.

3. The medical device positioner of claim 1, wherein the positioner surrounds an implantable device and temporarily or permanently positions the implantable device in the chest.

4. The positioner of claim 1, wherein the positioner has one or more attachment points on either or both of the first or second enclosures that allow for mating and/or attachment of the first enclosure to the second enclosure.

5. The positioner of claim 1, wherein the enclosures are comprised of a non-biological, biodegradable, or absorbable material.

6. The positioner of claim 1, wherein the enclosures are round and have the same circumference.

7. The positioner of claim 1, wherein the enclosures are round and a fixed circumference.

8. The positioner of claim 1, wherein the enclosure size is adjustable.

9. The positioner of claim 1, wherein the one or more enclosures are circular, oval, square, rectangular, triangular, elliptical, or a modification thereof.

10. The positioner of claim 4, wherein the attachment point comprises a tightening-type device.

11. The positioner of claim 4, wherein the attachment point comprises one or more of a peg and hole system, interlocking pegs, or a hinge.

12. The positioner of claim 1, wherein the wherein the attachment point comprises nested enclosures.

13. The positioner of claim 1, wherein the first and/or second enclosures contains attachment points that can are used to secure the enclosure in the body wherein the attachment points can be removed from the enclosure.

14. The positioner of claim 1, wherein the implantable device is one or more of a breast implant, a breast sizer, acellular dermal matrix, or an absorbable or non-absorbable mesh.

15. The positioner of claim 1, wherein the overlying mold is placed over the implantable device and attached to the first or second enclosure.

16. A method for positioning an implantable medical device in a human, wherein the implantable device is covered in a biologic mesh creating a wrapped device;
surrounding the wrapped device with a first enclosure;
positioning the first enclosure with wrapped device within the body;
mating the first enclosure with the wrapped device with a second enclosure that attaches to the first enclosure;
adhering the implantable device covered in biologic mesh to the body; and
removing the first and second enclosures from the body.

17. The method of claim 1, wherein the first enclosure is adhered within the body using sutures, adhesive and clamps and wherein the first enclosure is adhered in the body using attachment points integrated into the first enclosure.

18. The method of claim 1, wherein the circumference of the first and second enclosures is adjustable.

19. The method of claim 1, wherein the first and second enclosures are circular, oval, square, rectangular, triangular, elliptical, or a modification thereof.

20. The method of claim 1, wherein the first and/or second enclosure has one or more attachment points that allow for attachment of the closures to each other and wherein the attachment points are removable from the first and/or second enclosure.
